(19) United States
(12) Patent Application Publication
(10) Pub. No.: US 2025/0366943 A1
(43) Pub. Date: Dec. 4, 2025

(54) METHOD AND SYSTEM FOR CONTROLLING A SLAVE DEVICE, IN A MASTER-SLAVE ROBOTIC SYSTEM FOR SURGICAL TELEOPERATION, AT PHYSICAL LIMITS OF MOVEMENT OF THE SLAVE DEVICE

(71) Applicant: MEDICAL MICROINSTRUMENTS, INC., Wilmington, DE (US)

(72) Inventors: Antonio DI GUARDO, Pisa (IT); Emanuele RUFFALDI, Pisa (IT); Matteo TANZINI, Pisa (IT)

(21) Appl. No.: 18/869,237

(22) PCT Filed: May 25, 2023
(86) PCT No.: PCT/IB2023/055370

§ 371 Date: Nov. 25, 2024

(30) Foreign Application Priority Data
May 25, 2022 (IT) 102022000010883

(51) Int. Cl. A61B 34/35; A61B 34/00

(52) U.S. Cl. CPC A61B 34/35; A61B 34/74

(57) ABSTRACT

A method controls a robotic system slave device for medical/surgical teleoperation. The system includes a hand-held master device movable by an operator and controlling a slave device. If a nominal target pose having an orientation in a rotational space of the slave device is outside a slave device working region, and the nominal or modified target pose inside the working region, are inside the departure region, the slave device orientation is converged to the modified target pose. If the nominal and/or modified target poses are outside the departure region, rotational movement of the slave device is blocked until the nominal and modified target poses enter into the reentry region. When the nominal and modified target poses return to the reentry region, the slave device orientation is converged to the modified target pose, through a teleoperation phase, ending when the slave device orientation converges to the modified target pose.

### CROSS-REFERENCE TO RELATED APPLICATIONS

This application is the National Phase of International Application No. PCT/IB2023/055370, filed on May 25, 2023, which claims priority to Italian Patent Application No. 102022000010883, filed on May 25, 2022, the entire contents of which are incorporated into this application by reference.

FIELD OF APPLICATION

The present invention relates to a method and system for controlling a robotic system for medical or surgical teleoperation.

In particular, the invention relates to a method for controlling a slave device, controlled by a master device movable by an operator in a robotic system for medical or surgical teleoperation, close to physical limits of movement of the slave device, related in particular to rotational degrees of freedom, and related robotic system.

### PRIOR ART

Master devices having an appendage mechanically constrained to the operating console are generally known in the field of master-slave robotic systems for medical or surgical teleoperation. Typically, such an appendage comprises actuation motors which actuate the master device to limit the movement thereof under certain conditions.

The operating console typically further comprises a foot switch for transmitting control signals to the slave device in turn. Alternatively or in addition to the foot switch, a switch can be included on the body of the master device itself.

Master devices for medical or surgical teleoperation have also been suggested, which are mechanically directly constrained to one or more slave robotic arms for moving said one or more slave robotic arms by means of master-slave actuation kinematics.

Master devices for medical or surgical teleoperation mechanically constrained to the console by means of a cardan suspension (“gimbal”) are also known.

Otherwise, robotic systems for medical or surgical teleoperation are also known having master devices not mechanically constrained to the operating console of the robotic system (also called: “mechanically ungrounded”, “mechanically unconstrained”, “mechanically groundless”), i.e., of the type as shown for example in WO-2019-020407, WO-2019-020408, WO-2019-020409,WO-2021-161158, WO-2021-161185 and WO-2021-161177 in the name of the Applicant.

Another category of master devices is the non-actuated or “ungrounded” type, i.e., without feedback systems from the slave device which could physically limit the maneuverability thereof. Both master devices of the mechanically unconstrained type and master devices of the type constrained to the operating console can belong to this category, for example where a cardan support and stabilization joint (“gimbal”) is included. In a master device of the “ungrounded” type, without force feedback, and in mono-lateral teleoperation, there is a problem related to what occurs when the master device maps on a corresponding nominal target pose which is not reachable by the slave device, for example because it is outside an allowed workspace for the slave device.

In order to maintain high teleoperation usability, and to maintain intuitive operator behavior, the need arises to provide modified and improved control approaches and algorithms when the slave device is located close to the limits of the allowed workspace and/or when the nominal target pose is outside the aforesaid workspace of the slave device.

A mono-lateral teleoperation is given between, for example, a symmetrical N-fold type master device and a slave device (microsurgical instrument) in which there are degrees of freedom of a translational nature (generally 3 directions orthogonal to one another), degrees of freedom of a rotational nature (the space attitude of which can generally be described by 3 successive rotations), and possibly additional degrees describing the state of the microsurgical device, such as the “closure” (or grip).

It is assumed that the symmetrical N-fold master device has at least the same number of degrees of freedom as the controlled device. In this context, the mono-lateral teleoperation can be seen as an information flow between master device and slave device (as shown for example in FIG. 4).

Since the master device is unconstrained, there is no a priori fixed mapping between the positions of the master device and the slave device. Such a mapping is created in particular moments such as the entry into teleoperation, in which the movement of the slave device “couples” to that of the master device.

In a microsurgical context, with particular reference to rotational degrees of freedom, the unconstrained master device includes the possibility of uniquely associating the orientation of the master device with that of the slave device a priori. Such a mapping is generally 1-1.

However, any rotational misalignments between the master device and the non-recoverable slave device are a source of degradation of the performance of the robotic system.

In this regard, the presence of limits, with reference for example to the rotational degrees of freedom, requires a particular management of teleoperation close to such limits.

For example, the prior art document EP 3459429 A1 shows a robotic system for laparoscopic surgical teleoperation with a robotic surgical probe insertion control system configured to allow the simultaneous insertion of all the robotic probes in a coordinated manner based on a single command by the user.

Therefore, in master-slave robotic systems with both constrained and unconstrained master device, the need is felt to define an appropriate teleoperation behavior when the slave device is located close to such limits as well as to optimize the user experience during the change of the teleoperation paradigm.

The known solutions, in the technical field considered, do not allow satisfactorily solving the aforesaid problems and drawbacks.

Therefore, in the technical field considered, there is a strong need to control the enslaved movement of the slave device, depending on the master device, with contrivances and based on control algorithms such as to solve or at least mitigate the aforesaid problems and drawbacks.

In particular, the need is felt to be able to ensure the surgeon gripping at least one master control device of the type not constrained to the console and/or of the type without force feedback, an intuitive and smooth teleoperation session, even close the limits of the rotational workspace of the slave device, such as the physical limits of the rotational joints of the slave surgical instrument and/or the slave robotic manipulator.

In greater detail, where the current pose of the slave device results in one or more slave rotational joints being near the physical limits thereof, there is a need to continue teleoperation without interruption even if the pose commanded by the unconstrained master device and/or without force feedback would impose exceeding said physical limits.

### SUMMARY OF THE INVENTION

It is an object of the present invention to provide a method for controlling a slave device, controlled by an unconstrained master device movable by an operator close to physical limits of movement of the slave device, which allows at least partially obviating the drawbacks complained above with reference to the prior art, and responding to the aforementioned needs particularly felt in the technical field considered.

Such an object is achieved by a method for controlling a slave device of a robotic system for medical or surgical teleoperation, wherein said robotic system comprises at least one hand-held master device adapted to be moved by an operator, and at least one slave device adapted to be controlled by the at least one master device.

The method comprises:
defining a nominal target pose having a respective orientation in a rotational space of the at least one slave device, the at least one slave device having a respective working region belonging to the rotational space of the at least one slave device;
defining, in the rotational space of the at least one slave device, a modified target pose defined so that it is within the working region of the at least one slave device;
defining a departure region and a reentry region, centered on a current pose of the at least one slave device, wherein the departure region is a first subspace of the rotational space of the at least one slave device, and wherein the reentry region is a second subspace of the rotational space of the at least one slave device;
controlling the movement of the at least one slave device so that:
a) if the nominal target pose is outside said working region, and the nominal target pose and the modified target pose are inside said departure region, the orientation of the at least one slave device is controlled to converge to the modified target pose;
b) if at least one of the nominal target pose and the modified target pose is outside said departure region, the rotational movement of the at least one slave device is blocked until both the nominal target pose and the modified target pose enter into said reentry region;
c) when, after exiting the working region, both the nominal target pose and the modified target pose return to the reentry region, the orientation of the at least one slave device is controlled to converge to the modified target pose, through a slowed teleoperation phase, the slowed teleoperation phase ending when the orientation of the at least one slave device converges to the modified target pose.

It is also an object of the present invention to provide a robotic system for medical or surgical teleoperation, configured to be controlled by the aforesaid method.

### BRIEF DESCRIPTION OF THE DRAWINGS

Further features and advantages of the method according to the invention will become apparent from the following description of preferred embodiments, given by way of non-limiting indication, with reference to the accompanying drawings, in which:

FIG. 1 shows a master-slave robotic system for medical or surgical teleoperation, according to an embodiment of the present invention;

FIGS. 2 and 3 show in more detail, respectively, a master device and a slave device, included in the robotic system in FIG. 1, according to an embodiment of the present invention;

FIG. 4 shows an information flow in a diagrammatic and simplified manner in a master-slave robotic system, between master device and slave device;

FIGS. 5-12, 6a, 7a and 9a illustrate, by means of diagrammatic graphic representations, operating conditions which can occur during the implementation of the method in accordance with the present invention, relevant for the purposes of describing the method according to the invention.

It is worth noting that equal or similar elements in the figures will be indicated by the same numeric or alphanumeric references.

### DETAILED DESCRIPTION

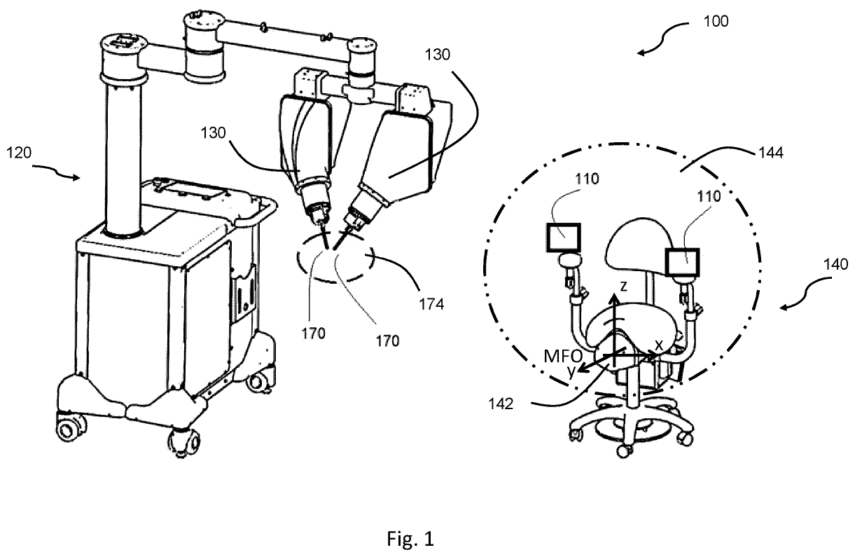

With reference to FIGS. 1-12, 6a, 7a and 9a, a method for controlling a slave device 170 of a robotic system 100 for medical or surgical teleoperation, hereinafter also robotic system or simply system, is now described.

The robotic system 100 comprises at least one hand-held master device 110 and adapted to be moved by an operator 150.

The robotic system 100 further comprises at least one slave robotic assembly 120 comprising at least one slave device 170 (or slave surgical instrument 170) adapted to be controlled by the at least one master device 110.

The slave robotic assembly 120 can further comprise at least one non-sterile manipulator 130 comprising one or more motorized actuators and adapted to be controlled by the at least one master device 110, in which the at least one slave device 170 can be operatively and detachably connected to the at least one manipulator 130.

Preferably, the at least one device 170 is sterile and is connected to the manipulator 130 by interposing a sterile barrier (not shown).

The at least one slave device 170 can comprise a distal articulated end moved by tendons or actuating cables operatively connectable to the motorized actuators of the at least one non-sterile manipulator 130 of the slave robotic assembly 120.

In accordance with an embodiment, the slave robotic assembly 120 comprises two slave devices 170 (or slave surgical instruments 170) working in the same shared workspace.

The at least one master device 110 is preferably a master device of “ungrounded” type, without force feedback, for mono-lateral teleoperation. For example, the at least one master device 110 can thus be a master mechanically constrained to an operating console 140 while being of the ungrounded type without force feedback, for mono-lateral teleoperation.

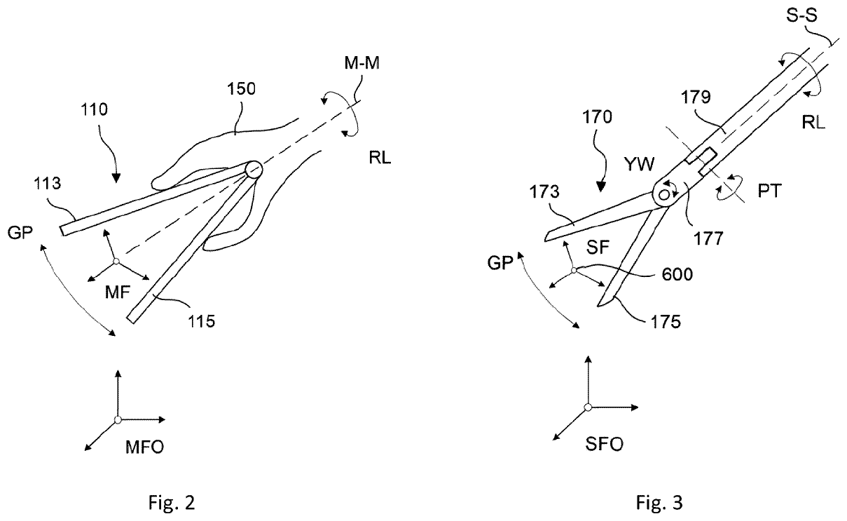

Alternatively, the at least one master device 110 can be a master device of the type mechanically unconstrained to the operating console 140, as shown for example in FIG. 2.

With reference to FIG. 1, the operating console 140 can comprise a tracking field generator 142, e.g., an electromagnetic field emitter, to enable the position and/or orientation of the at least one master device 110 within the generated tracking field 144 to be detected.

For example, the origin of the master global reference system (MFO, “master frame origin”) can coincide with the location of the tracking field generator 142.

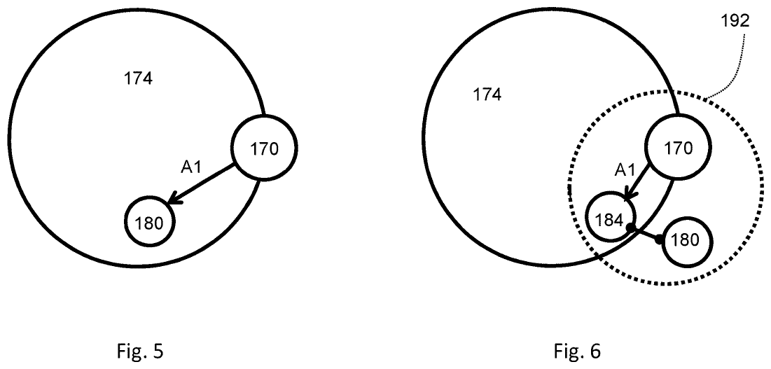

With reference again to the method in accordance with the present invention and also to FIGS. 5-12, 6a, 7a and 9a, the method comprises a step of defining a nominal target pose 180 having a respective orientation in a rotational space of the at least one slave device 170.

The at least one slave device 170 has a respective working region 174 belonging to the rotational space of the at least one slave device 170.

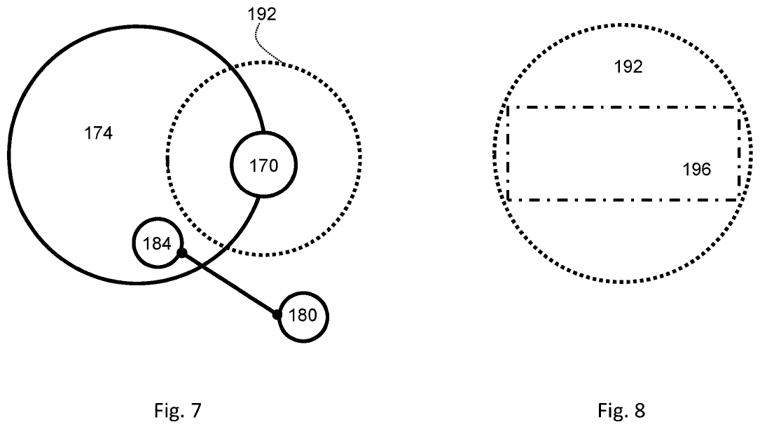

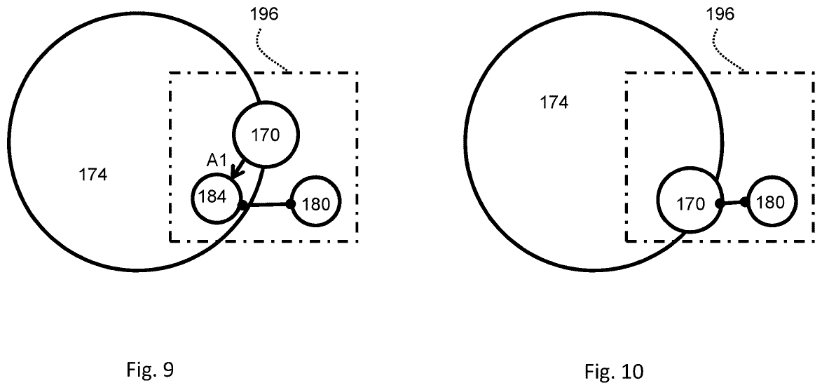

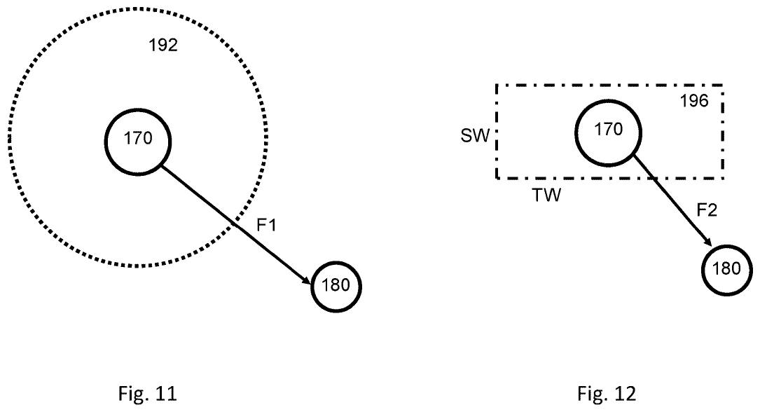

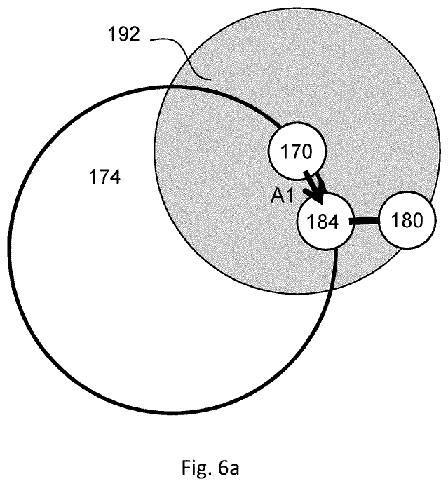

The method further comprises a step of defining in the rotational space of the at least one slave device 170 a modified target pose (“proxy”) 184 defined so that it is within the working region 174 of the at least one slave device 170 (see for example FIGS. 6, 6a, 7, 7a, 9, and 9a).

The method further comprises a step of defining a departure region 192 (see for example FIGS. 6, 6a, 7, 7a, 8, and 11) and a reentry region 196 (see for example FIGS. 8, 9, 9a, 10, and 12), centered on a current pose of the at least one slave device 170.

The departure region 192 is a first subspace of the rotational space of the at least one slave device 170.

The reentry region 196 is a second subspace of the rotational space of the at least one slave device 170.

The departure region 192, centered on the current pose of the at least one slave device, is therefore that rotation attraction subspace (for example, roll-pitch-yaw) defined to evaluate the removal of the nominal target pose 180 from said working region 174 in a step of exiting/removing the nominal target pose 180 from the working region 174.

The reentry region 196, centered on the current pose of the at least one slave device, is instead that rotation attraction subspace (for example roll-pitch-yaw) defined for the approach of the nominal target pose 180 to said working region 174 in a step of approaching the nominal target pose 180 to the working region after the nominal target pose 180 has exited the working region.

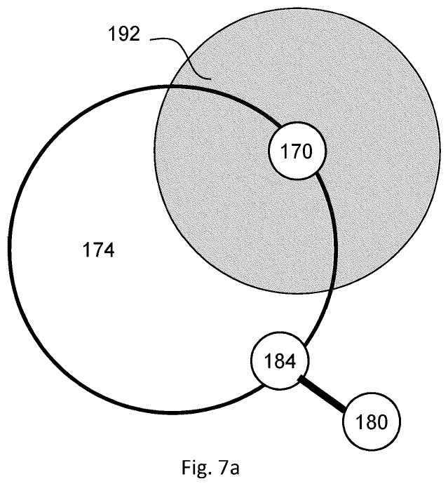

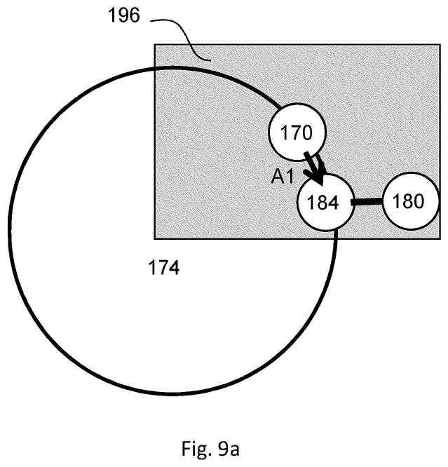

The method further comprises a step of controlling the movement of the at least one slave device 170 so that:
a) if the nominal target pose 180 is outside said working region 174, and the nominal target pose 180 and the modified target pose 184 are inside said departure region 192 (as shown diagrammatically, for example, in FIGS. 6 and 6a), the orientation of the at least one slave device 170 is controlled to converge to the modified target pose 184 (as shown diagrammatically in FIGS. 6 and 6a with an arrow Al depicting the convergence movement of the at least one slave device 170 to the modified target pose);
b) if at least one of the nominal target pose 180 and the modified target pose 184 is outside said departure region 192 (as shown diagrammatically for example in FIGS. 7 and 7a, in which the nominal target pose 180 is outside said departure region 192), the rotational movement of the at least one slave device 170 is blocked until both the nominal target pose 180 and the modified target pose 184 enter into said reentry region 196 (as shown diagrammatically, for example, in FIGS. 9 and 9a);
c) when, after exiting the working region 174, both the nominal target pose 180 and the modified target pose 184 re-enter the reentry region 196 (as shown diagrammatically, for example, in FIGS. 9 and 9a), the orientation of the at least one slave device 170 is controlled to converge to the modified target pose 184 (as shown diagrammatically in FIGS. 9 and 9a with the arrow Al depicting the convergence movement of the at least one slave device 170 to the modified target pose), through a slowed teleoperation phase. “Slowed teleoperation” means a teleoperation in which the dynamics of the slave device are slowed down, i.e., a scaling of the speed and rotational acceleration of the slave device.

It should therefore be noted that the slowdown envisaged in the “slowed teleoperation” does not refer to a scaling of the positions of the slave device.

The slowed teleoperation phase ends when the orientation of the at least one slave device 170 converges to the modified target pose 184 (as shown diagrammatically, for example, in FIG. 10, where only the at least one slave device 170 is shown as fully overlapping the modified target position 184 in FIGS. 9 and 9a).

In accordance with an embodiment of the method, the step of controlling comprises a step of controlling the movement of the at least one slave device 170 so that:
if the nominal target pose 180 is within said working region 174 (as diagrammatically shown in FIG. 5), the orientation of the at least one slave device 170 is controlled to converge towards said nominal target pose 180 (as diagrammatically shown in FIGS. 9 and 9a with the arrow Al depicting the convergence movement of the at least one slave device 170 to the modified target pose).

In greater detail, the orientation of the at least one slave device 170 is controlled to converge towards said nominal target pose 180 for example through a normal dynamics of the teleoperation phase.

“Normal dynamics” means that the medical or surgical teleoperation is performed at a set “normal” teleoperation speed, i.e., neither slowed down nor accelerated.

In accordance with an implementation, the orientation of the at least one slave device 170 is evaluated by evaluating the orientation of a virtual control point 600 associated with or integral with the slave surgical instrument 170.

The virtual control point 600, shown diagrammatically in FIG. 3, represents the origin of a local reference system SF (“Slave Frame”) of the at least one slave device 170.

In accordance with an embodiment of the method, in combination with any of the foregoing, the step of controlling comprises a step of controlling the movement of the at least one slave device 170 so that:
from when the controlled teleoperation phase is terminated, the orientation of the at least one slave device 170 is controlled to converge to the modified target pose 184.

In greater detail, the orientation of the at least one slave device 170 is controlled to converge to the modified target pose 184 e.g., through a normal dynamic, defined above, of the teleoperation phase.

In accordance with an embodiment of the method, in combination with any of the foregoing, the departure region 192 is defined as the set of all nominal or modified target poses in which a respective first error function F1 is applied between the nominal target pose 180 or the modified target pose 184 of the at least one slave device 170 and the current pose of the at least one slave device 170. Such a first error function F1 is lower than a set first threshold.

The first error function F1, also described in more detail below, is diagrammatically depicted with an arrow in FIG. 11.

In accordance with this embodiment of the method, the reentry region 196 is defined as the set of all nominal or modified target poses in which a respective second error function F2 is applied between the nominal target pose 180 or the modified target pose 184 of the at least one slave device and the current pose of the at least one slave device 170. Such a second error function F2 is lower than a set second threshold.

The second error function F2, also described in greater detail below, is diagrammatically depicted with an arrow in FIG. 12.

In accordance with an embodiment of the method, in combination with any of the foregoing, not shown in the figures, the departure region 192 is a static region which externally surrounds the working region 174 of the at least one slave device 170.

In accordance with an embodiment of the method, alternative to the preceding one, the departure region 192 is a dynamic region, variable as a function of the current pose of the at least one slave device 170, and extending at least partly outside the working region 174 of the at least one slave device 170.

In accordance with an embodiment of the method, in combination with any of the foregoing and shown diagrammatically in FIG. 8, the reentry region 196 is contained within the retraction region 192, so as to evaluate an approach of the nominal target pose 180 from said working region 174 in a step of approaching the nominal target pose 180 to the working region 174 after the nominal target pose 180 has exited the working region 174.

In greater detail, the reentry region 196 is tightly contained within the departure region 192.

“Tightly contained” means that the reentry region 196 is below and entirely contained within the departure region 192.

For example, as shown diagrammatically in FIG. 8, the boundary of the reentry region 196 is entirely contained within the departure region 192.

In accordance with an embodiment of the method, in combination with any of the foregoing, the modified target pose 184 is defined from the nominal target pose 180 as follows:

if the nominal target pose 180 is within the working region 174, the modified target pose 184 is coincident with the nominal target pose 180.

if the nominal target pose 180 is outside said working region 174, the modified target pose 184 is that at the border of the working region 174 as close as possible to the nominal target pose 180.

Moreover, in accordance with a further embodiment, if there are more positioning conditions of the nominal target pose 180 with respect to the working region 174, the modified target pose 184 is the one closest to the current pose of the at least one slave device 170.

In accordance with an embodiment of the method, in combination with any of the foregoing, the rotational space of the at least one slave device 170 is a rotational space limited in SO(3) parameterizable by said rotational coordinates comprising three Eulerian coordinates “Roll” (indicated by reference sign RL in FIG. 3), “Pitch” (indicated by reference sign PT in FIG. 3) and “Yaw” (indicated by reference sign YW in FIG. 3).

In accordance with an embodiment of the method, in combination with any of the foregoing in which said first error function F1 and said second error function F2 are included, the slow teleoperation phase comprises:
controlling the dynamics of the at least one slave device 170 so that the speeds of the at least one slave device 170, with reference to the rotational coordinates, are lower than speed values of the at least one slave device 170 provided in the teleoperation and/or inversely proportional to said first error function F1 and said second error function F2 expressed in terms of solid angle.

In accordance with an embodiment of the method, in combination with the foregoing, the method comprises a step of:
signaling to the operator that the slowed teleoperation phase is in progress, in which the dynamics of the at least one slave device 170 are controlled so that the speeds of the at least one slave device 170 are lower than the speed values of the at least one slave device 170 provided in the medical teleoperation and inversely proportional to said first error function F1 and said second error function F2 expressed in terms of solid angle.

In accordance with an embodiment, the step of signaling is carried out persistently, for example by emitting an audio signal (“beep”) which lasts for the duration of the slowed teleoperation.

In accordance with an embodiment of the method, in combination with any of the foregoing, the nominal target pose 180 of the at least one slave device 170 comprises rotational degrees of freedom and further degrees of freedom with respect to the rotational degrees of freedom.

Said further degrees of freedom are controlled in a manner which does not depend on and is not affected by said determination of the orientation of the nominal target pose 180 of the at least one slave device 170, with reference to said rotational coordinates, with respect to said working region 174.

In accordance with an embodiment of the method, in combination with the preceding one, the control of said further degrees of freedom also occurs if the movement of the at least one slave device 170 with reference to said rotational coordinates has been blocked.

In accordance with an embodiment of the method, in combination with any of the foregoing in which said further degrees of freedom are included, said further degrees of freedom comprise translational degrees of freedom.

In accordance with this embodiment, if the orientation of the nominal target pose 180 is outside of said working region 174 and is outside of said departure region 192, and thus the rotational coordinates are blocked, the method further comprises:
blocking and/or preventing the movement of the at least one slave device 170 in speed with reference to said translational degrees of freedom.

In accordance with an embodiment of the method, as an alternative to the preceding one, in which said further degrees of freedom comprise translational degrees of freedom and in which, if the orientation of the nominal target pose 180 is outside said working region 174 and is outside said departure region 192, and thus the rotational coordinates are blocked, the method further comprises:
limiting the movement of the at least one slave device 170 in speed with reference to said translational degrees of freedom.

In accordance with an embodiment of the method, in combination with any of the foregoing in which said first error function F1 and said second error function F2 are included, the method comprises a step of calculating said first error function F1 and/or said second error function F2 as a solid angle between the orientation of the nominal target pose 180 of the at least one slave device 170 and the orientation of the current pose of the at least one slave device 170.

In accordance with an embodiment of the method, in combination with the preceding one, said first error function F1 and/or said second error function F2 are calculated by twist/swing decomposition of the angle between nominal target pose 180 of the slave device 170 and the current pose of the at least one slave device 170.

The swing angle (diagrammatically indicated by reference sign SW in FIG. 12) is a solid angle between the main direction of the nominal target pose 180 of the at least one slave device 170 and the main direction of the orientation of the current pose of the at least one slave device 170.

The swing angle represents a rotation about a swing axis orthogonal to the main directions of orientation of the at least one master device 110 and the at least one slave device 170.

The twist angle (diagrammatically indicated by reference sign TW in FIG. 12) is a solid angle about the main direction of orientation of the at least one slave device 170 necessary to align with the nominal target pose 180, applying a rotation of the swing angle about said swing axis to the orientation of the at least one slave device 170.

In accordance with an embodiment of the method, in combination with any one of the foregoing in which said first error function and said second error function are included, the first error function F1 and the second error function F2 are scalar functions, and the first threshold and the second threshold are scalar values.

In accordance with an embodiment of the method, in combination with any one of the foregoing in which said first error function F1 and said second error function F2 are included, the first threshold and the second threshold are equal.

In accordance with an embodiment of the method, in combination with any of the foregoing in which said first error function F1 and said second error function F2 are included and alternatively to the preceding case, the second threshold is lower than the first threshold.

In accordance with an embodiment of the method, in combination with any of the foregoing in which said first error function F1 and said second error function F2 are included:
the at least one slave device 170 is of the “end effector” type;
the at least one master device 110 is of the “2-fold symmetrical” type.

For example, with reference to FIG. 3, the at least one slave device 170 of the “end-effector” type preferably comprises a pair of terminal gripping and/or cutting links 173, 175 (“instrument tips”) articulated to each other defining an opening/closing degree of freedom GRIP (indicated by reference sign GP in FIGS. 2 and 3), and preferably further comprises at least one further link 177 supporting said pair of terminal links 173, 175 and which can be articulated with respect to a frame 179 of the at least one slave device 170, forming an articulated end-effector.

Moreover, with reference to FIG. 2, again by way of example, the at least one master device 110 comprises two rigid parts 113, 115 constrained to rotate with respect to each other about a common axis, in the manner of a clamp or forceps (as shown for example in FIG. 2), in which said two rigid parts 113, 115 are substantially symmetrical with respect to the longitudinal axis M-M of the master device 110.

If a mono-lateral teleoperation is given between the at least one master device 110, in this embodiment, i.e., of the “symmetrical N-fold” type, and the at least one slave device 170 (microsurgical instrument), there are degrees of freedom of a translational nature (generally 3 mutually orthogonal directions), degrees of freedom of a rotational nature (the space attitude of which can generally be described by 3 successive rotations) and possibly additional degrees describing the state of the microsurgical device, such as the “closure” (or grip).

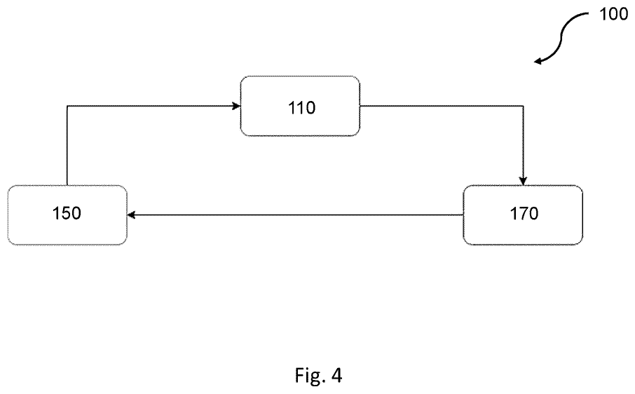

As mentioned above, in this context, the mono-lateral teleoperation can be seen as an information flow between the at least one master device 110 and the at least one slave device 170 (surgical instrument), as shown for example in FIG. 4.

In this embodiment, said first error function F1 and said second error function F2 are calculated on both nominal target mutually spaced apart by an angle equal to 180° about the main direction of the at least one master device 110.

The nominal target pose 180 used in the reentry phase is the one with less angular distance with respect to the slave device 170 (known as “Flip active” mode).

In accordance with an embodiment, in combination with the preceding one, the method comprises a step of blocking the orientation of the at least one master device of the “2-fold symmetrical” type (known as “Flip freeze” mode).

In greater detail, it is the property of axial symmetry with respect to the axis of the at least one master device 110 which necessarily has an effect on the detection and transmission of the rotational degree of freedom of roll RL to the at least one slave device 170, i.e., there are two 180° rotated poses which are substantially equal.

As shown for example in FIG. 3, the at least one slave device 170 can have a degree of freedom of roll RL about the slave roll axis S-S which can extend along the longitudinal direction of the frame 179, e.g., a spindle or rod, of the at least one slave device 170.

In accordance with an embodiment of the method, in combination with any of the foregoing in which the step of signaling is included, said step of signaling comprises, alternatively or in combination, steps of:
providing an audio signal (“beeping”);
providing a video signal (light change);
providing a message on a video terminal.

In accordance with an embodiment of the method, in combination with any of the foregoing, the transition between the step of blocking the rotational movement of the at least one slave device 170 and the step of controlling the orientation of the at least one slave device 170 to converge to the modified target pose 184, through a slowed teleoperation phase, is performed automatically without the direct intervention of the operator 150 on any button and/or pedal.

In accordance with an embodiment of the method, in combination with any of the foregoing, the step of defining a nominal target pose 180 having a respective orientation in a rotational space of the at least one slave device 170 comprises a step of calculating the orientation of the nominal target pose 180 from the orientation of the at least one master device 110 within a rotational space of the at least one master device 110.

In an embodiment, alternatively or in combination with the preceding one, the step of defining in the rotational space of the at least one slave device 120 a modified target pose (proxy) 184, comprises a step of calculating the modified target pose (proxy) from the nominal target pose 180 as a projection on the working region 174 of the at least one slave device 170, belonging to the rotational space of the at least one slave device 170, using a projection function so that the modified target pose is within the working region 174 of the at least one slave device 170, in which if the nominal target pose 180 belongs to the working region 174 of the at least one slave device 170, the nominal target pose 180 and the modified target pose 184 are coincident.

In an embodiment, alternatively or in combination with the foregoing, the step of defining a departure region 192 and a reentry region 196, centered on the current pose of the at least one slave device 170, comprises that the departure region 192 is a first subspace of the rotational space of the at least one slave device 170, and is defined to evaluate the removal of the nominal target pose 180 from said working region in a step of exiting/removing the nominal target pose 180 from the working region 174.

In an embodiment, alternatively or in combination with the foregoing, the reentry region 196 is defined to evaluate an approach of the nominal target pose 180 to said working region 174 in a step of approaching the nominal target pose 180 to the working region once the nominal target pose 180 has exited the working region 174.

Still referring to FIGS. 1-10, and in particular FIGS. 1-3, and as mentioned above, the robotic system 100 for medical or surgical teleoperation included in the present invention is described below.

Such a robotic system 100 comprises at least one hand-held master device 110 adapted to be moved by an operator 150.

The system 100 comprises at least one slave robotic assembly 120 comprising at least one slave device 170 (or slave surgical instrument 170) adapted to be controlled by the at least one master device 110.

The system 100 further comprises a control unit configured to control the at least one slave device 170, during a teleoperation, based on the movements of the at least one master device (110).

The control unit is further configured to carry out the following actions:
defining a nominal target pose 180 having a respective orientation in a rotational space of the at least one slave device 170, the at least one slave device 170 having a respective working region 174 belonging to the rotational space of the at least one slave device 170;
defining in the rotational space of the at least one slave device 170 a modified target pose (“proxy”) 184 defined so that it is within the working region 174 of the at least one slave device 170 (as shown, for example, in FIGS. 6, 6a, 7, 7a, 9 and 9a);
defining a departure region 192 (see for example FIGS. 6, 6a, 7, 7a, 8, and 11) and a reentry region 196 (see for example FIGS. 8, 9, 9a, 10, and 12), centered on the current pose of the at least one slave device 170, in which the departure region 192 is a first subspace of the rotational space of the at least one slave device 170, and in which the reentry region 196 is a second subspace of the rotational space of the at least one slave device 170.

The control unit of the robotic system 100 is further configured to control the movement of the at least one slave device 170 so that:
a) if the nominal target pose 180 is outside said working region 174, and the nominal target pose 180 and the modified target pose 184 are inside said departure region 192 (as shown diagrammatically, for example, in FIGS. 6 and 6a), the orientation of the at least one slave device 170 is controlled to converge to the modified target pose 184 (as shown diagrammatically in FIGS. 6 and 6a with the arrow Al depicting the convergence movement of the at least one slave device 170 to the modified target pose 184);
b) if at least one of the nominal target pose 180 and the modified target pose 184 is outside said departure region 192 (as shown diagrammatically for example in FIGS. 7 and 7a, in which the nominal target pose 180 is outside said departure region 192), the rotational movement of the at least one slave device 170 is blocked until both the nominal target pose 180 and the modified target pose 184 enter into the aforesaid reentry region 196 (as shown diagrammatically, for example, in FIGS. 9 and 9a);
c) when, after exiting the working region 174, both the nominal target pose 180 and the modified target pose 184 re-enter the reentry region 196 (as shown diagrammatically, for example, in FIGS. 9 and 9a), the orientation of the at least one slave device 170 is controlled to converge to the modified target pose 184 (as shown diagrammatically in FIGS. 9 and 9a with the arrow A1 depicting the convergence movement of the at least one slave device 170 to the modified target pose), through a slowed teleoperation phase, the slowed teleoperation phase ending when the orientation of the at least one slave device 170 converges to the modified target pose 184 (as shown diagrammatically for example in FIG. 10, where only the at least one slave device 170 is shown as completely overlapping the modified target position 184 in FIGS. 9 and 9a).

According to several possible embodiments of the robotic system 100, the control unit is configured to carry out a method for controlling a slave device according to any one of the embodiments shown in this description.

Still referring to the aforesaid FIGS. 1-12, 6a, 7a and 9a, a method for controlling a slave device 170 of a robotic system 100 for medical or surgical teleoperation, hereinafter also robotic system or simply system, further forms the subject of the present invention.

The robotic system 100 comprises at least one hand-held master device 110 and adapted to be moved by an operator 150.

The at least one master device 110 has already been described above.

The robotic system 100 further comprises at least one slave robotic assembly 120 comprising at least one slave device 170 (or surgical instrument 170 adapted to be controlled by the master device 110).

The slave robotic assembly 120 has already been described above.

With reference again to the method in accordance with this embodiment and also FIGS. 5-12, 6a, 7a and 9a, the method comprises a step of defining a nominal target pose 180 having a respective orientation in a rotational workspace of the at least one slave device 170 defined by rotational coordinates representative of a rotational metric, corresponding to a respective pose of the at least one master device 110 having a respective orientation in a rotational workspace of the at least one master device 110 defined by rotational coordinates representative of a rotational metric.

The orientation of the nominal target pose 180 of the at least one slave device 170 and said orientation of the pose of the at least one master device 110 are characterized by rotational coordinates in the rotational workspace of the at least one slave device 170.

The method further comprises a step of determining the orientation of the nominal target pose 180 of the at least one slave device 170, with reference to said rotational coordinates, with respect to at least one working region (workspace) 174.

The method further comprises a step of controlling the movement of the at least one slave device 170 so that:
if the orientation of the nominal target pose 180, with reference to said rotational coordinates, is within said workspace 174 (as shown, for example, in FIG. 5), following until reaching, by the at least one slave device 170, the orientation of the nominal target pose 180 through a normal teleoperation phase (already defined above) (as shown diagrammatically in FIG. 5 with the arrow A1 representative of the convergence movement of the at least one slave device 170 to the nominal target pose 180).

The method further executes the step of controlling the movement of the at least one slave device 170 so that:
if the orientation of the nominal target pose 180, with reference to rotational coordinates, is outside said working region (workspace) 174 (as shown, for example, in FIGS. 6, 6a, 7, 7a, 9, 9a and 10), defining a first error function F1 and a second error function F2 (both already described above) between the orientation of the nominal target pose 180 of the at least one slave device 170 and the orientation of the current pose of the at least one slave device 170 and carrying out the following action:
if said first error function F1 assumes a value lower than a set first threshold, controlling the movement of the at least one slave device 170 so that the rotational coordinates of the at least one slave device 170 converge towards an orientation of a pose.

In an embodiment, in combination with the preceding one, the comparison between the value of said first error function F1 and the established first threshold is carried out to verify the presence or absence of the nominal target pose 180 within a first departure region 192 defined, in the rotational space of the at least one slave device 170, around said current pose of the at least one slave device 170 (as shown, for example, in FIGS. 6, 6a, 7, 7a, 9, 9a and 10), in which said first departure region 192 is defined to evaluate the removal of the nominal target pose 180 from said working region 174 in a phase of exiting/removing the nominal target pose 180 from the working region 174. 20)

In accordance with an embodiment, in combination with any of the two preceding ones, the comparison between the value of said second error function F2 and the set second threshold is carried out to verify the presence or absence of the nominal target pose 180 within a second reentry region 196 defined in the rotational space of the at least one slave device 170 around said current position of the at least one slave device 170, in which said second reentry region 196 is defined to evaluate an approach of the nominal target pose 180 from said working region 174 in a step of approaching the nominal target pose 180 from the working region 174 after the nominal target pose 180 has exited the working region.

In an embodiment, in combination with the preceding one, the second reentry region 196 is a dynamic region, variable as a function of the current pose of the at least one slave device 170, and extending at least in part outside the working region 174 of the at least one slave device 170 (as shown, for example, in FIGS. 9, 9a, and 10).

As can be seen, the object of the present invention is fully achieved.

In fact, the method according to the present invention controls the enslaved movement of the slave device, depending on the master device, with contrivances and based on control algorithms such as to solve or at least mitigate the problems and drawbacks encountered in the state of the art.

In particular, the method controls a slave device, controlled by a master device movable by an operator, close to physical limits of movement of the slave device, in an accurate and timely manner.

By virtue of the suggested solutions, where the nominal target pose 180, having an orientation thereof in the rotational space of the slave device, is defined by movement of the master device unconstrained by the user, and where the nominal target pose 180 is not feasible (not reachable) by the slave device because it is outside the rotational space of the slave device, then the method (and related system) defines a modified target pose 184 which is substantially the projection of the target pose 180 in the rotational space of the slave device, for example the orientation closest to the target pose 180 which is inside the rotational space of the slave device.

Thereby it is thus possible to have a slowed (“smooth”) teleoperation without hiccups or interruptions even where the pose commanded by the master device (i.e., said nominal target pose 180) is outside the space of the rotations of the slave device or beyond the physical limits of one or more rotational joints of the slave surgical instrument.

Moreover, to allow the control of the slave device also close to the limits of the rotational space of the slave device, for example where the nominal target pose 180 controlled by the unconstrained master device is outside said rotational space of the slave device and thus not reachable by the slave device, by virtue of the suggested solutions, two rotation attraction subspaces (e.g., roll-pitch-yaw) are defined around the current pose of the slave device called the departure region 192 and the reentry region 196.

Thereby, the method (and related system) selectively blocks or enables the movement of the slave device based on the location of the nominal target pose 180 and the modified target pose 184 with respect to the aforesaid rotational attraction subspaces.

In fact, the current pose of the slave device 170 can already be close to the rotational physical limits, so that the definition of such subspaces of attraction of departure and reentry allows maintaining the intuitive and slowed (“smooth”) teleoperation, slowing down the rotational dynamics (i.e., the speed or acceleration) of the slave device if necessary.

Preferably, as mentioned above, with the prediction of a state of “slowed teleoperation” i.e., of slowing down the dynamics of the slave device, it is intended to indicate a scaling of the speeds and rotational accelerations of the slave device, and it is not intended to indicate the scaling of the positions.

The state of slowed teleoperation allows slowing down the rotational dynamics (speed/acceleration) of the slave device close to the limits of the rotational space thereof (for example limit stroke of the rotational joints of the articulated surgical instrument), avoiding water hammers and allowing an intuitive control by the user even close to said limits where certain conditions are verified with respect to the defined attraction subspaces.

The intuitiveness of use finds particular benefits from the fact that the method (and related system) maintains the teleoperation condition without interrupting it even where the controlled pose is outside the slave rotational space as well as for the prediction of the slowed dynamics which allows minimizing situations in which the slave device does not move in response to a command given by the unconstrained master control device and/or without force feedback.

By virtue of the suggested solutions, a master control device not constrained to the operating console, which therefore potentially has no limit or rotational physical limit switch, is allowed to finely control the slave device even in orientation, avoiding interrupting the teleoperation even if the controlled pose (nominal target pose 180) is outside the rotational space of the slave device.

The modified target pose 184 is preferably the closest pose to the nominal target pose 180 within the slave rotational space and within the boundaries of said attraction subspaces (departure region 192, reentry region 196).

Those skilled in the art may make changes and adaptations to the embodiments of the method and related system described above or can replace elements with others which are functionally equivalent in order to meet contingent needs without departing from the scope of the following claims. Each of the features described as belonging to a possible embodiment can be implemented irrespective of the other embodiments described.

1. A method for controlling a slave device of a robotic system for medical or surgical teleoperation, wherein said robotic system comprises at least one hand-held master device adapted to be moved by an operator, and at least one slave device adapted to be controlled by the at least one master device, the method comprising:
defining a nominal target pose having a respective orientation in a rotational space of the at least one slave device, the at least one slave device having a respective working region belonging to the rotational space of the at least one slave device;
defining, in the rotational space of the at least one slave device, a modified target pose defined so that the modified target pose is within the working region of the at least one slave device;
defining a departure region and a reentry region, centered on a current pose of the at least one slave device, wherein the departure region is a first subspace of the rotational space of the at least one slave device, and wherein the reentry region is a second subspace of the rotational space of the at least one slave device;
controlling the movement of the at least one slave device so that:
a) if the nominal target pose is outside said working region, and the nominal target pose and the modified target pose are inside said departure region, the orientation of the at least one slave device is controlled to converge to the modified target pose;
b) if at least one of the nominal target pose and the modified target pose is outside said departure region, rotational movement of the at least one slave device is blocked until both the nominal target pose and the modified target pose enter into said reentry region;
c) when, after exiting the working region, both the nominal target pose and the modified target pose return to the reentry region, the orientation of the at least one slave device is controlled to converge to the modified target pose, through a slowed teleoperation phase, the slowed teleoperation phase ending when the orientation of the at least one slave device converges to the modified target pose.

2. The method according to claim 1, wherein controlling the movement of the at least one slave device comprises controlling the movement of the at least one slave device so that:
if the nominal target pose is within said working region, the orientation of the at least one slave device is controlled to converge towards said nominal target pose.

3. The method according to claim 1, wherein controlling the movement of the at least one slave device comprises controlling the movement of the at least one slave device so that:
from when the controlled teleoperation phase is terminated, the orientation of the at least one slave device is controlled to converge to the modified target pose.

4. The method according to
the departure region is defined as the a set of all the nominal or modified target poses in which a respective first error function is applied between the nominal target pose or the modified target pose of the at least one slave device and the current pose of the at least one slave device, such a the first error function being lower than a set first threshold;
the reentry region is defined as a set of all the nominal or modified target poses in which a respective second error function is applied between the nominal target pose or the modified target pose of the at least one slave device and the current pose of the at least one slave device, such a the second error function being lower than a set second threshold.

5. The method according to any one of the preceding claims claim 1, wherein said departure region is a static region externally surrounding the working region of the at least one slave device.

6. The method according to claim 1, wherein said departure region is a dynamic region, varying as a function of the current pose of the at least one slave device, and extending at least partially outside the working region of the at least one slave device.

7. The method according to any one of the preceding claims claim 1, wherein said reentry region is contained within the departure region, so as to evaluate an approach of the nominal target pose from said working region in approaching the nominal target pose to the working region after the nominal target pose has exited the working region.

8. The method according to claim 1, wherein the modified target pose is defined from the nominal target pose as follows:
if the nominal target pose is within the working region, the modified target pose is coincident with the nominal target pose;
if the nominal target pose is outside said working region, the modified target pose is at the border of the working region as close as possible to the nominal target pose.

9. The method according to any one of the preceding claims claim 1, wherein said rotational space of the at least one slave device is a rotational space limited in SO(3) parameterizable by said rotational coordinates comprising three Eulerian coordinates Roll, Pitch and Yaw.

10. The method according to claim 1, wherein said slowed teleoperation phase comprises:
controlling dynamics of the at least one slave device so that speeds of the at least one slave device, with reference to the rotational coordinates, are lower than speeds of the at least one slave device provided in the teleoperation and/or inversely proportional to said first error function and said second error function expressed in terms of solid angle.

11. (canceled)

12. The method according to claim 1, wherein the nominal target pose of the at least one slave device comprises rotational degrees of freedom and further degrees of freedom with respect to the rotational degrees of freedom, wherein said further degrees of freedom are controlled in a manner which does not depend on and is not affected independent of and unaffected by said determination of the orientation of the nominal target pose of the at least one slave device, with reference to said rotational coordinates, with respect to said working region.

13. The method according to claim 12, wherein control of said further degrees of freedom also occurs if the movement of the at least one slave device with reference to said rotational coordinates has been blocked.

14. (canceled)

15. The method according to claim 12, wherein said further degrees of freedom comprise translational degrees of freedom, and wherein, if the orientation of the nominal target pose is outside said working region and is outside said departure region, and thus the rotational coordinates are blocked, the method further comprises:
limiting the movement of the at least one slave device in speed with reference to said translational degrees of freedom.

16. The method according to claim 4, comprising:
calculating said first error function and/or said second error function as a solid angle between the orientation of the nominal target pose of the at least one slave device and the current pose orientation of the at least one slave device.

17. (canceled)

18. The method according to claim 4, wherein the first error function and the second error function are scalar functions, and the first threshold and the second threshold are scalar values.

19-20. (canceled)

21. The method according to claim 1, wherein:
the at least one slave device comprises two terminal links defining a degree of opening/closing freedom;
the at least one master device comprises a twofold symmetrical device;
said first error function and said second error function are calculated on both of the nominal target poses mutually spaced apart by an angle equal to 180° about a main direction of the at least one master device, the nominal target pose used in the reentry phase having a smaller angular distance with respect to the slave device.

22. The method according to claim 21, comprising:
blocking the orientation of the at least one master device of the comprises a twofold symmetrical device.

23. (canceled)

24. The method according to claim 1, wherein the switching between blocking the rotational movement of the at least one slave device and controlling the orientation of the at least one slave device to converge to the modified target pose, through a slowed teleoperation phase, is performed automatically without the direct intervention of the operator on a button and/or pedal.

25. The method according to claim 1, wherein:
defining a nominal target pose having a respective orientation in a rotational space of the at least one slave device comprises calculating the orientation of the nominal target pose from the orientation of the at least one master device within a rotational space of the at least one master device;
and/or wherein:
defining a modified target pose in the rotational space of the at least one slave device comprises a step of calculating the modified target pose from the nominal target pose as a projection on a working region of the at least one slave device, belonging to the rotational space of the at least one slave device, using a projection function so that the modified target pose is within the working region of the at least one slave device, wherein if the nominal target pose belongs to the working region of the at least one slave device, the nominal target pose and the modified target pose are coincident;
and/or wherein
defining a departure region and a reentry region, centered on the current pose of the at least one slave device, and wherein the departure region is a first subspace of the rotational space of the at least one slave device, and is defined to evaluate the removal of the nominal target pose from said working region in exiting/removing the nominal target pose from the working region,
and/or wherein
the reentry region is defined to evaluate an approach of the nominal target pose to said working region of approaching the nominal target pose to the working region once the nominal target pose has exited the working region.

26. The method according to claim 1, wherein the at least one master device is a non-force feedback device, for single-sided teleoperation; and/or wherein the at least one master device is mechanically unconstrained to a console of the robotic system.

27. A robotic system for medical or surgical teleoperation, comprising:
at least one hand-held master device adapted to be moved by an operator;
at least one slave device adapted to be controlled by the at least one master device;
a control unit configured to control the at least one slave device, during a teleoperation, based on movements of the at least one master device,
wherein the control unit is further configured to:
define a nominal target pose having a respective orientation in a rotational space of the at least one slave device, the at least one slave device having a respective working region belonging to the rotational space of the at least one slave device,
define, in the rotational space of the at least one slave device, a modified target pose defined so that the modified target pose is within the working region of the slave device;
define a departure region and a reentry region, centered on the current pose of the at least one slave device, wherein the departure region is a first subspace of the rotational space of the at least one slave device, and wherein the reentry region is a second subspace of the rotational space of the at least one slave device;
controlling the movement of the at least one slave device so that:
a) if the nominal target pose is outside said working region, and the nominal target pose and the modified target pose are inside said departure region, the orientation of the at least one slave device is controlled to converge to the modified target pose;
b) if at least one of the nominal target pose and the modified target pose is outside said departure region, the rotational movement of the at least one slave device is blocked until both the nominal target pose and the modified target pose enter into the reentry region;
c) when, after exiting the working region, both the nominal target pose and the modified target pose return to the reentry region, the orientation of the at least one slave device is controlled to converge to the modified target pose, through a slowed teleoperation phase, the slowed teleoperation phase ending when the orientation of the at least one slave device converges to the modified target pose.

28-51. (canceled)
